(19) United States
(12) Patent Application Publication
(10) Pub. No.: US 2023/0377682 A1
(43) Pub. Date: Nov. 23, 2023

(54) PEPTIDE BINDING MOTIF GENERATION

(71) Applicant: NEC Laboratories America, Inc., Princeton, NJ (US)

(72) Inventors: Renqiang Min, Princeton, NJ (US); Hans Peter Graf, South Amboy, NJ (US)

(21) Appl. No.: 18/319,803

(22) Filed: May 18, 2023

Related U.S. Application Data
(60) Provisional application No. 63/344,081, filed on May 20, 2022.

(51) Int. Cl. G16B 15/30; G16B 20/30; G16B 40/20

(52) U.S. Cl. CPC G16B 15/30; G16B 20/30; G16B 40/20

(57) ABSTRACT

Methods and systems for peptide generation include training a peptide mutation policy neural network using reinforcement learning that includes a peptide presentation score as a reward. New peptides are generated using the peptide mutation policy. A binding motif of a major histocompatibility complex is calculated using the new peptides. Library peptides are screened in accordance with the binding motif.

### RELATED APPLICATION INFORMATION

This application claims priority to U.S. Patent Application No. 63/344,081, filed on May 20, 2022, incorporated herein by reference in its entirety.

### BACKGROUND

### Technical Field

The present invention relates to binding peptide identification and, more particularly, to reinforcement learning models that generate binding peptides.

### Description of the Related Art

Immunotherapy aims at boosting a patient's immune system against pathogens and tumor cells. The immune response is triggered when immune cells recognize foreign peptides, presented by major histocompatibility complex (MHC) proteins on a cell's surface. To be recognized, the foreign peptides are bound to MHC Class I proteins. The resulting peptide-MHC complexes interact with T cell receptors. These interactions can be leveraged to generate peptide-based vaccines to prevent disease.

However, identification of peptides that bind to specific MHC proteins is a significant challenge, as the search space of all possible peptides is intractably large.

### SUMMARY

A method for peptide generation includes training a peptide mutation policy neural network using reinforcement learning that includes a peptide presentation score as a reward. New peptides are generated using the peptide mutation policy. A binding motif of a major histocompatibility complex (MHC) is calculated using the new peptides. Library peptides are screened in accordance with the binding motif.

A system for peptide generation includes a hardware processor and a memory that stores a computer program. When executed by the hardware processor, the computer program causes the hardware processor to train a peptide mutation policy neural network using reinforcement learning that includes a peptide presentation score as a reward. A plurality of new peptides is generated using the peptide mutation policy. A binding motif of an MHC is calculated using the plurality of new peptides. A plurality of library peptides is screened in accordance with the binding motif.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a diagram of a bond between a peptide and a major histocompatibility complex (MHC), in accordance with an embodiment of the present invention;

FIG. 2 is a block/flow diagram of a method of training a mutation policy and using the mutation policy to generate binding peptides for MHCs, in accordance with an embodiment of the present invention;

FIG. 3 is a block/flow diagram of a method of generating and administering a peptide-based vaccine, in accordance with an embodiment of the present invention;

FIG. 4 is a diagram of an exemplary neural network architecture which may be used to form part of a mutation policy, in accordance with an embodiment of the present invention;

FIG. 5 is a diagram of an exemplary deep neural network architecture which may be used to form part of a mutation policy, in accordance with an embodiment of the present invention; and

FIG. 6 is a block diagram of a computing device that may store and execute computer program code to train a mutation policy, to generate peptides, and/or to perform peptide screening, in accordance with an embodiment of the present invention.

### DETAILED DESCRIPTION OF PREFERRED EMBODIMENTS

Foreign peptides that bind to a given major histocompatibility complex (MHC) may be identified using a reinforcement learning model. The model learns a mutation policy that optimizes peptides by changing amino acids step-by-step, such that the mutated peptides are likely to be presented by a given MHC protein. The generated motifs are robust, with random initial peptides leading to identical motifs after stepwise mutations, and are highly correlated to experimentally derived motifs.

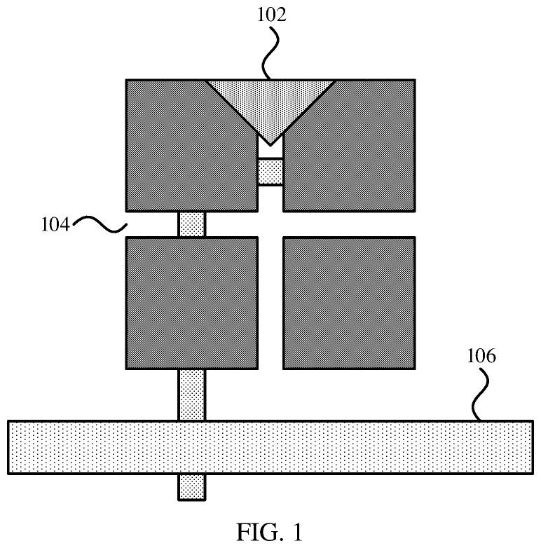

Referring now to FIG. 1, a diagram of a peptide-MHC protein bond is shown. A peptide 102 is shown as binding with an MHC protein 104, with complementary two-dimensional interfaces of the figure suggesting complementary shapes of these three-dimensional structures. The MHC protein 104 may be attached to a cell surface 106.

An MHC is an area on a DNA strand that codes for cell surface proteins that are used by the immune system. MHC molecules are used by the immune system and contribute to the interactions of white blood cells with other cells. For example, MHC proteins impact organ compatibility when performing transplants and are also important to vaccine creation.

A peptide, meanwhile, may be a portion of a protein. When a pathogen presents peptides that are recognized by a MHC protein, the immune system triggers a response to destroy the pathogen. Thus, by finding peptide structures that bind with MHC proteins, an immune response may be intentionally triggered, without introducing the pathogen itself to a body. In particular, given an existing peptide that binds well with the MHC protein 104, a new peptide 102 may be automatically identified according to desired properties and attributes.

Interactions between peptides and MHCs play a role in cell-mediated immunity, regulation of immune responses, and transplant rejection. Prediction of peptide-protein binding helps guide the search for, and design of, peptides that may be used in vaccines and other medicines. Given a library of known peptides, new peptide sequences can be generated using mutation policies. The resulting mutated peptides may be within a threshold number of amino acid differences from the library of peptides. When the library of peptides is derived from a particular pathogen, such as a virus or tumor sample, the mutated peptides can be used to target the specific pathogen or tumor. This makes it possible to, for example, identify and target a specific cancer for an individual.

Thus, given a particular genome (e.g., sequenced from a tumor cell), peptide sequences may be extracted to generate a library of peptides that uniquely identifies the pathogen. By targeting this library, peptides can be screened/selected that bind to MHCs that are present on cell surfaces, so that immune responses can be triggered to kill the pathogen or tumor cells.

Toward that end, a deep neural network may be trained using a training dataset to predict a peptide presentation score given an MHC allele sequence and a peptide sequence. The peptide presentation score may be, e.g., a combination of peptide-MHC binding affinity and an antigen processing score.

Based on the trained peptide presentation model, deep reinforcement learning may be used to generate binding peptide motifs. The pretrained presentation score prediction model may be used to define reward functions starting from random peptides. The deep reinforcement learning system may be trained to learn good peptide mutation policies by transforming a given random peptide into a peptide with a high presentation score.

When applying a reinforcement learning system to this process, the “state” may be interpreted as being a given MHC allele sequence and peptide sequence, while the “action” may be interpreted as an edit to the peptide sequence. Such an edit may replace a current amino acid at a determined position of the peptide sequence with a new amino acid.

The amino acid sequences may be embedded using a one-dimensional convolutional layer on top of concatenated amino acid embeddings and fully connected layers of a neural network model to generate an MHC allele representation. A bi-directional long-short term memory (LSTM) layer may further process the amino acid embeddings to obtain a peptide representation. A deep policy network may then learn the conditional probability of the different actions may be learned given the state. At each time step, if the peptide presentation score of the mutated peptide based on an action is increased more than a threshold, it may be assigned a positive reward value, and otherwise it may be assigned a negative reward value.

A peptide scoring model may be trained to accept as input a peptide ρ and an MHC protein m and to generate an output score r(ρ, m) that represents a binding affinity between the peptide ρ and the protein m, in particular representing the probability that the peptide ρ will be presented on a cell surface by the protein m. In some cases, the presentation score may be a composite score of an antigen processing prediction and a binding affinity prediction, where the former predicts a probability for a peptide to be delivered by the transporter associated with antigen processing protein complex into the endoplasmic reticulum, where the peptide can bind to MHC proteins.

A mutation policy network may also be trained. The mutation policy network guides how peptide sequences are modified. As will be described in greater detail below, this policy network guides the reinforcement learning system, taking as an input a peptide and an MHC protein and outputting a modification or “mutation” of the peptide. The policy network selects the mutation with the goal of improving the presentation score of the mutated peptide to the MHC protein. A library of peptides may be sampled, and this sampling may be performed randomly. The sampled peptides may then be mutated according to the mutation policy.

Within this framework, a peptide may be represented as a sequence of amino acids ρ=<o₁, o₂, . . . , oₗ>, where o is one of a set of natural amino acids and l is the length of the sequence, for example ranging between 8 and 15. A reinforcement learning agent explores the peptide mutation environment for high-presentation peptide generation. Thus, given a pair of inputs (ρ, m), the reinforcement learning agent explores and exploits the peptide mutation environment by repeatedly mutating the peptide and observing the resulting presentation score. The agent thereby learns the mutation policy π(·) to iteratively mutate amino acids of any given peptide to generate a high presentation score. Thus, a peptide mutation environment and a mutation policy network are determined.

The peptide mutation environment enables the reinforcement learning agent to perform trial-and-error peptide mutations to gradually refine its mutation policy, through tuning the parameters of the mutation policy network. During learning, the reinforcement learning agent keeps mutating peptides and determining their presentation scores as a reward signal. The rewards help reinforce the agent's mutation behaviors, with those mutation behaviors that produce high presentation scores being encouraged.

The mutation environment includes a state space, an action space, and a reward function. The state includes the current mutated peptide and the MHC protein. The action and the reward represent the mutation action that may be taken by the reinforcement learning agent, resulting in a new presentation score for the mutated peptide, respectively.

The state of the environment may be defined as sₜ at a time t for a pair (ρ, m). The MHC protein may be represented as a pseudo-sequence, for example with thirty-four amino acids, each being in potential contact with the bound peptide within a distance of, e.g., 4.0 Å. With a peptide of length l and an MHC protein, the state sₜ may be represented as the tuple sₜ=(E^{p}, E^{m}), where E^{p} and E^{m} are the encoding matrices of the peptide and the MHC protein, respectively. The state s₀ may be initialized by sampling a peptide sequence from a library and using an MHC class I protein. During training, any appropriate peptide sequence and MHC protein may be used. The terminal state s_{T} may be defined as the state with a maximum time step T or having a presentation score greater than a predetermined threshold α. When the terminal state s_{T} is reached, the mutation of the peptide may be halted.

A multi-discrete action space may be defined to optimize the peptide by replacing one amino acid with another. At a time t, given a peptide ρₜ, the action for the reinforcement learning agent may be to determine the position of the amino acid oᵢ being replaced and then to predict a type of new amino acid for that position. The reward function guides the optimization of the reinforcement learning agent, where only the terminal states can receive rewards from the peptide mutation environment. The final reward may be determined as r(ρ_{T}, m), with the peptide PT being in the terminal state s_{T}.

In one exemplary reward function, a score may be a composite score the antigen processing prediction and the binding affinity prediction. The former predicts the probability for a peptide to be delivered by the transporter associated with antigen processing protein complex into the endoplasmic reticulum, where the peptide can bind to MHC proteins. The latter predicts the binding strength between the peptide and MHC proteins. Higher presentation scores indicate higher antigen processing and binding affinity scores, and indicate higher probabilities for peptides to be presented on the cell surface by the given MHC proteins.

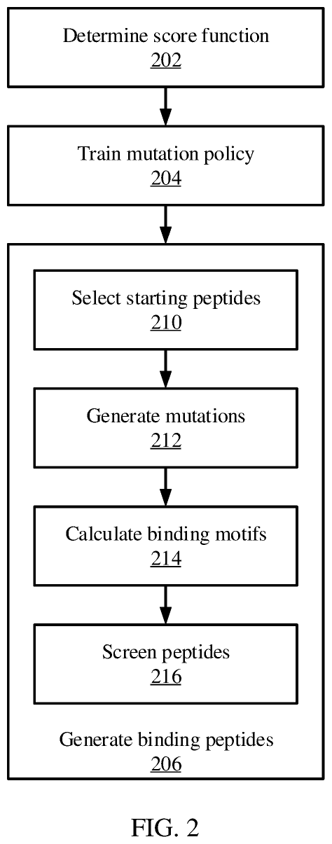

Referring now to FIG. 2, a method for generating binding peptides is shown. Block 202 determines a score function, where the output of the score function characterizes a quality of a binding between a peptide sequence and an MHC allele sequence. This score may be implemented as a presentation score, providing a combination of peptide-MHC binding affinity and antigen processing scores. The score function may be implemented as, for example, a deep neural network that is trained on a public peptide dataset or may reflect a pre-trained scoring model.

A peptide mutation policy is trained 204 based on the scoring function, for example using a deep reinforcement learning system. The peptide mutation policy takes a peptide sequence as an input and generates an output peptide with one or more changes—referred to herein as mutations. Using the score function to define a reward function and starting from a peptide sequence, a deep reinforcement learning system is trained to learn good peptide mutation policies that transform a given input peptide into a peptide with a high presentation score.

Block 206 uses the score function and the trained peptide mutation policy to generate binding peptides based on input peptides. The input peptides may be randomly sampled from any appropriate dataset in block 210. Using the sampled peptide(s) as input, block 212 applies the trained peptide mutation policy to generate new peptide sequences.

Block 214 calculates a binding motif of all MHCs of interest, including uncommon MHCs that do not have significant amounts of experimental data. The binding motif may include a position weighted matrix, with the probabilities of amino acids at each motif position. Using the binding motif of a given MHC, the peptides in a sequencing library may be screened by block 216.

In a first example of peptide screening, the weighted block substitution matrix (BLOSUM) representations of amino acids may be calculated for each position in the binding motif, for example using the amino acid probabilities in the position weight matrix at each position to weight the BLOSUM representations of amino acids. A weighted sum may then be calculated as the final representation for each position. A pairwise Euclidean distance can then be used between the calculated motif BLOSUM representation and the BLOSUM representation of a peptide for screening. In a second example of peptide screening, a log-likelihood of a peptide can be calculated under the position weight matrix of the motif.

To learn the peptide mutation policy in block 204, a reinforcement learning agent learns to mutate amino acids in an input peptide sequence, one amino acid at each step, with the goal of maximizing the presentation score of the mutated peptide. Both the peptide and the MHC protein may be encoded into a distributed embedding space, and then a mapping between the embedding space and the mutation policy may be learned by a gradient descent optimization.

Multiple encoding methods may be used to represent the amino acids within the peptide sequences and the MHC proteins. Each amino acid may be represented by concatenating encoding vectors e^{B} from a BLOSUM, e⁰ from a one-hot matrix, and e⁰ from a learnable embedding matrix. Thus, e=e^{B}⊕e⁰⊕e^{D} where e∈ [symbol] =(d=B+O+D). This achieves good binding prediction performance on peptide-MHC proteins. The encoding matrices E^{ρ} and E^{m} of the peptide ρ and the MHC protein m may then be represented as E^{ρ}={e₁; . . . ; eₗ} ∈ [symbol] and E^{m}={e₁; . . . ; e_{M}}∈ [symbol] , respectively, with M being a number of available amino acids and l is the length of the peptide.

Each amino acid oᵢ in a peptide sequence p may be embedded into a continuous latent vector hᵢ using, for example, a one-layer bidirectional LSTM as:

\vec{h}ᵢ,\vec{c}ᵢ=LSTM(eᵢ,\vec{h}ᵢ₋₁,\vec{c}ᵢ₋₁,\vec{W}^{ρ})

[symbol]

ᵢ, [symbol] ᵢ=LSTM(eᵢ, [symbol] ᵢ₊₁, [symbol] ᵢ₊₁, [symbol] )

hᵢ=hᵢ⊕ [symbol] ᵢ

where [symbol] and \vec{h} are hidden state vectors of the i^{th} amino acid, \vec{c} and [symbol] are memory cell states of the i^{th} amino acid, \vec{h}₀, [symbol] ₗ, \vec{c}₀, and [symbol] ₗ are initialized with random values, and \vec{W}^{ρ} and [symbol] are learnable parameters of the LSTM in the forward and backward direction, respectively. The embedding of the peptide sequence may be defined as the concatenation of hidden vectors at two ends: h^{ρ}=\vec{h}ₗ⊕ [symbol] ₀.

To embed an MHC protein into a continuous latent vector, the encoding matrix E^{m} MAY BE flattened into a vector m. The continuous latent embedding h^{m} may be learned as:

h^{m}=W₁^{m} ReLu(W₂^{m}m)

where ReLU(·) is a rectified linear unit activation function and [symbol] ( [symbol] =1,2) are learnable parameter matrices.

At each time step t, the peptide sequence ρₜ may be optimized by predicting the mutation of one amino acid with the latent embeddings h^{ρ}^{ₜ} and h^{m}. Specifically, the amino acid oᵢ may be selected from ρₜ as the amino acid to be replaced. For each amino acid oᵢ in the peptide sequence, the score of the replacement may be predicted as:

ƒ^{c}(oᵢ)=(w^{c})^{T}(ReLU(W₁^{c}hᵢ+W₂^{c}h^{m}))

where hᵢ is the hidden latent vector of oᵢ, and w^{c} and [symbol] are the learnable vector and matrices, respectively. The likelihood of replacing amino acid oᵢ with another amino acid can be measured by looking at its context in hᵢ and the MHC protein h^{m}. The amino acid to be replaced may be determined by sampling from the distribution with normalized scores. The type of amino acid that replaces of may be determined as:

ƒ^{d}(o)=softmax(W₁^{d}×ReLu(W₂^{d}hᵢ+W₃^{d}h^{m})

where [symbol] ( [symbol] =1,2,3) are learnable matrices and where softmax(·) converts a twenty-dimensional vector into probabilities over the twenty amino acid types. The amino acid type may then be determined by sampling from the distribution of probabilities of amino acid types, excluding the original amino acid type oᵢ.

The objective function for learning the mutation policy may be defined as:

$${max}_{θ} {L}^{CLIP} ( θ ) = {t}_{} [ min ( {r}_{t} ( θ ) {\hat{A}}_{t} , clip ( {r}_{t} ( θ ) , 1 - ϵ , 1 + ϵ ) {\hat{A}}_{t} ) ]$$

where [symbol] is an expectation with respect to a time step t (e.g., the average over all time steps), θ is the set of learnable parameters of the policy network and

$${r}_{t} ( θ ) = \frac{{π}_{θ} ( {a}_{t} \vert {s}_{t} )}{{π}_{{θ}_{old}} ( {a}_{t} \vert {s}_{t} )}$$

is the probability ratio between the action under current policy π^{θ} and the action under the previous policy π_{θ}_{_{old}}. The ratio rₜ(θ) is clipped to avoid moving rₜ outside the interval [1−ϵ, 1+ϵ]. The term Âₜ is the advantage at time step t, computed with a generalized advantage estimator, measuring how much better the selected actions are than others on average:

Âₜ=δₜ+(γλ)δₜ₊₁+ . . . +(γλ)^{T-t+1}δ_{T-1}

where γ∈(0,1) is a discount factor determining the importance of future rewards, δₜ=rₜ+γV₍ₛ_{ₜ}₊₁₎−V(sₜ) is the temporal difference error, V(sₜ) is a value function, and λ∈(0,1) is a parameter used to balance the bias and variance of V(sₜ).

The value function V(sₜ) may use a multi-layer perceptron to predict the future return of current state sₜ from the MHC embedding h^{m} and the peptide embedding h^{ρ}. The objective function of V(·) may be defined as:

$${max}_{θ} {L}^{V} ( θ ) = {t}_{} [ {( V ( {s}_{t} ) - {\hat{R}}_{t} )}^{2} ]$$

where \hat{R}ₜ=Σᵢ₌ₜ₊₁^{T}γ^{i-t}rᵢ is a rewards-to-go value. Because only the final rewards are used (e.g., rᵢ=0∀i≠T), \hat{R}ₜ may be calculated as \hat{R}=γ^{T-t}r_{T}. The entropy regularization loss H(θ) may also be used to encourage exploration of the policy.

To stabilize the training and to improve performance, an expert policy πₑₚₜ may be derived from existing data. For each MHC protein m with sufficient binding peptide data, the amino acid distributions <ρ₁ (o|m), ρ₂ (o|m), . . . , ρₗ(o|m)> of peptides with length l may be determined. Given a peptide ρ, the position I may be selected as follows:

$${p}_{ept}^{c} ( p , m ) = {arg max}_{i} ( {p}_{i} ( o = {\hat{o}}_{i} ) - {p}_{i} ( o = {o}_{i} \vert m ) )$$

where ôᵢ is the most popular amino acid on position i. In other words,

$${p}_{i} ( o = {\hat{o}}_{i} \vert m ) = {max}_{o} ( {p}_{i} ( o \vert m ) ) .$$

After determining the position, the amino acid can be sampled from the distribution o′ᵢ˜ρᵢ(o|m). For an MHC protein without experimental data, the distances can be calculated with all of the MHCs with data, for example using a block substitution matrix, and actions can be sampled from the amino acid distributions with the most similar MHC.

The expert policy can be used to pre-train the policy network. The objective function for pre-training can minimize the following cross-entropy loss:

$${max}_{θ} {L}^{PRE} ( θ ) = {}_{s \sim S} [ {i \sim {π}_{ept}^{c}}_{} [ log ( {π}_{θ}^{c} ( i \vert s ) ) ] + {o \sim {π}_{ept}^{d}}_{} [ log ( {π}_{θ}^{d} ( o \vert s ) ) ] ]$$

where S denotes the state space and π_{θ}^{c} and π_{θ}^{d} are, respectively, parameterized by ƒ^{c} and ƒ^{d}, which are the policy networks for selecting the position and the amino acid for mutation. In addition to pre-training the policy network, actions can be sampled at the beginning of training using the expert policy, and the trajectories can be used with expert actions to update the policy network.

To increase the diversity of generated peptides, a non-deterministic policy can be used to produce diverse actions. Such a policy can increase the exploration over a large state space and can thereby find diverse good actions.

Entropy regularization can be included in the objective function to promote exploration. To explicitly enforce the policy's learning of diverse actions, a diversity-promoting experience buffer may be used to store trajectories that could result in qualified peptides. At each iteration, the visited state-action pairs of mutation trajectories for qualified peptides can be added to the buffer. The state-action pairs may be maintained with infrequent actions, and those with frequent actions can be removed to ensure that the buffer is not dominated by the frequent actions. A batch of state-action pairs with infrequent actions can be sampled from the buffer.

A cross-entropy loss L^{B} defined over the batch of state-action pairs with infrequent actions can then be included in the final objective function, to encourage the policy network to reproduce those infrequent actions that could induce high rewards:

$${max}_{θ} L ( θ ) = - {L}^{CLIP} ( θ ) + {α}_{1} {L}^{V} ( θ ) + {α}_{2} {L}^{B} ( θ ) + {α}_{3} H ( θ )$$

where H is the entropy of the policy network, and α₁, α₂, α₃ are predetermined coefficients.

Based on this trained DRL system with pretrained peptide mutation policies generate binding peptides are generated in block 212 from randomly sampled peptides. Block 214 calculates the binding motif (position weighted matrix, the probabilities of amino acids at each motif position) of all MHCs, including those uncommon MHCs that don't have many experimental data. Using the generated motifs of given MHCs, block 216 can rapidly screen all peptides in a sequencing library to identify neoantigens. This screening, being based on binding motifs, is robust and considers different peptide variations/mutations in the peptide library, which provides results that are superior to a single interaction score predicted by a classifier.

Real motifs may be characterized from experimental data, with an exemplary database including 149 human MHC proteins and 309,963 peptides in an experimental dataset. For computed motifs, a predetermined number (e.g., 1,000) of peptides may be generated for each of the human MHC proteins. The generated peptides with a presentation score below a predetermined threshold (e.g., 0.75) may be excluded due to their low binding affinity.

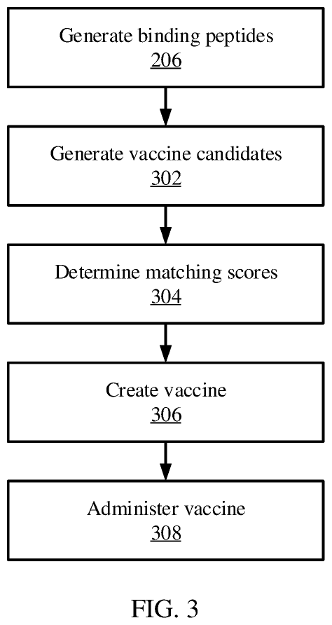

Referring now to FIG. 3, a method for treating an illness is shown. Block 206 generates a set of binding peptides for the MHC proteins, as described above. For a given illness, such as a viral infection, block 302 generates a set of peptide vaccine candidates, for example identifying peptides that may be presented by the infectious agent. When the infectious agent is in a human body, the MHCs may use these peptides to recognize the pathogen and trigger an immune response.

Block 304 uses the binding motifs from block 306 to determine matching scores for the vaccine candidates. These matching scores represent a binding affinity between the vaccine candidates and the MHC protein and reflect the peptide's ability to generate an immune response that will target the pathogen. Based on the matching scores, block 306 creates a vaccine by, e.g., generating neoantigens that incorporate a selected peptide vaccine candidate. Block 308 then administers the vaccine to prevent the illness.

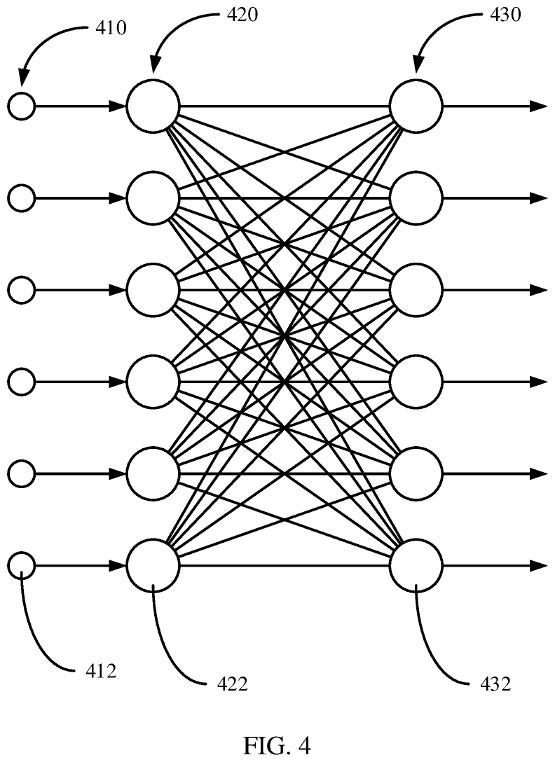

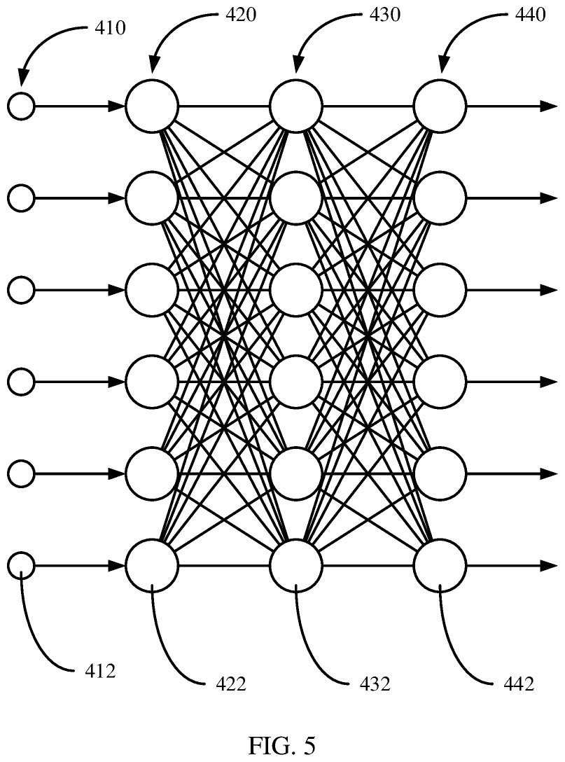

Referring now to FIGS. 4 and 5, exemplary neural network architectures are shown, which may be used to implement parts of the present models. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the inputted data belongs to each of the classes can be outputted.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 420 of source nodes 422, and a single computation layer 430 having one or more computation nodes 432 that also act as output nodes, where there is a single computation node 432 for each possible category into which the input example could be classified. An input layer 420 can have a number of source nodes 422 equal to the number of data values 412 in the input data 410. The data values 412 in the input data 410 can be represented as a column vector. Each computation node 432 in the computation layer 430 generates a linear combination of weighted values from the input data 410 fed into input nodes 420, and applies a non-linear activation function that is differentiable to the sum. The exemplary simple neural network can perform classification on linearly separable examples (e.g., patterns).

A deep neural network, such as a multilayer perceptron, can have an input layer 420 of source nodes 422, one or more computation layer(s) 430 having one or more computation nodes 432, and an output layer 440, where there is a single output node 442 for each possible category into which the input example could be classified. An input layer 420 can have a number of source nodes 422 equal to the number of data values 412 in the input data 410. The computation nodes 432 in the computation layer(s) 430 can also be referred to as hidden layers, because they are between the source nodes 422 and output node(s) 442 and are not directly observed. Each node 432, 442 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the inputted data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 432 in the one or more computation (hidden) layer(s) 430 perform a nonlinear transformation on the input data 412 that generates a feature space. The classes or categories may be more easily separated in the feature space than in the original data space.

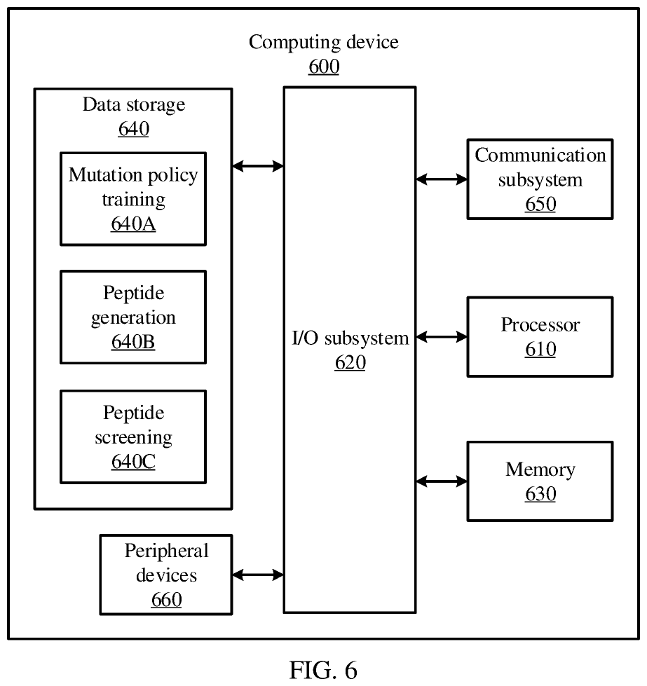

Referring now to FIG. 6, an exemplary computing device 600 is shown, in accordance with an embodiment of the present invention. The computing device 600 is configured to perform classifier enhancement.

The computing device 600 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 600 may be embodied as one or more compute sleds, memory sleds, or other racks, sleds, computing chassis, or other components of a physically disaggregated computing device.

As shown in FIG. 6, the computing device 600 illustratively includes the processor 610, an input/output subsystem 620, a memory 630, a data storage device 640, and a communication subsystem 650, and/or other components and devices commonly found in a server or similar computing device. The computing device 600 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 630, or portions thereof, may be incorporated in the processor 610 in some embodiments.

The processor 610 may be embodied as any type of processor capable of performing the functions described herein. The processor 610 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 630 may be embodied as any type of volatile or non-volatile memory or data storage capable of performing the functions described herein. In operation, the memory 630 may store various data and software used during operation of the computing device 600, such as operating systems, applications, programs, libraries, and drivers. The memory 630 is communicatively coupled to the processor 610 via the I/O subsystem 620, which may be embodied as circuitry and/or components to facilitate input/output operations with the processor 610, the memory 630, and other components of the computing device 600. For example, the I/O subsystem 620 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 620 may form a portion of a system-on-a-chip (SOC) and be incorporated, along with the processor 610, the memory 630, and other components of the computing device 600, on a single integrated circuit chip.

The data storage device 640 may be embodied as any type of device or devices configured for short-term or long-term storage of data such as, for example, memory devices and circuits, memory cards, hard disk drives, solid state drives, or other data storage devices. The data storage device 640 can store program code 640A for performing training the mutation policy network, 640B for generating peptides using the mutation policy, and/or 640C for screening the generated peptides. The communication subsystem 650 of the computing device 600 may be embodied as any network interface controller or other communication circuit, device, or collection thereof, capable of enabling communications between the computing device 600 and other remote devices over a network. The communication subsystem 650 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 600 may also include one or more peripheral devices 660. The peripheral devices 660 may include any number of additional input/output devices, interface devices, and/or other peripheral devices. For example, in some embodiments, the peripheral devices 660 may include a display, touch screen, graphics circuitry, keyboard, mouse, speaker system, microphone, network interface, and/or other input/output devices, interface devices, and/or peripheral devices.

Of course, the computing device 600 may also include other elements (not shown), as readily contemplated by one of skill in the art, as well as omit certain elements. For example, various other sensors, input devices, and/or output devices can be included in computing device 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 600 are readily contemplated by one of ordinary skill in the art given the teachings of the present invention provided herein.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for peptide generation, comprising:
training a peptide mutation policy neural network using reinforcement learning that includes a peptide presentation score as a reward;
generating a plurality of new peptides using the peptide mutation policy;
calculating a binding motif of a major histocompatibility complex (MHC) using the plurality of new peptides; and
screening a plurality of library peptides in accordance with the binding motif.

2. The method of claim 1, wherein calculating the binding motif of the MHC includes comprising generating a plurality of binding motifs for a plurality of respective MHCs, wherein screening includes screening in accordance with the plurality of binding motifs.

3. The method of claim 1, wherein training the peptide mutation policy neural network maximizes an objective function as:
$${max}_{θ} {L}^{CLIP} ( θ ) = {t}_{} [ min ( {r}_{t} ( θ ) {\hat{A}}_{t} , clip ( {r}_{t} ( θ ) , 1 - ϵ , 1 + ϵ ) {\hat{A}}_{t} ) ]$$
where θ represents parameters of the peptide mutation policy neural network, [symbol] ₜ is an expectation with respect to a time step t, rₜ(θ) is a probability ratio between an action under a current policy and an action under a previous policy, Âₜ is an average at time step t, clip(·) is a clipping function, and E is a size of a clipping interval.

4. The method of claim 1, wherein training the peptide mutation policy neural network includes pre-training using an expert policy.

5. The method of claim 1, wherein screening the plurality of library peptides includes determining pairwise Euclidean distances between block substitution matrix (BLOSUM) representations of the plurality of library peptides and a BLOSUM representation of the binding motif.

6. The method of claim 1, wherein screening the plurality of library peptides includes determining log-likelihoods of the plurality of library peptides under a weighted position of the binding motif.

7. The method of claim 1, wherein generating the plurality of new peptides includes sampling a random starting peptide and applying a change to the random starting peptide according to the peptide mutation policy.

8. The method of claim 1, wherein training the peptide mutation policy neural network includes changing an input peptide sequence as an action and determining a reward for the action based on the peptide presentation score of the changed input peptide sequence.

9. The method of claim 1, further comprising comparing the screened plurality of library peptides to a candidate vaccine peptide to determine how the candidate vaccine peptide binds to the MHC.

10. The method of claim 9, further comprising creating a vaccine based on the candidate vaccine peptide and administering the vaccine to prevent an illness.

11. A system for peptide generation, comprising:
a hardware processor; and
a memory that stores a computer program which, when executed by the hardware processor, causes the hardware processor to:
train a peptide mutation policy neural network using reinforcement learning that includes a peptide presentation score as a reward;
generate a plurality of new peptides using the peptide mutation policy;
calculate a binding motif of a major histocompatibility complex (MHC) using the plurality of new peptides; and
screen a plurality of library peptides in accordance with the binding motif.

12. The system of claim 11, wherein the computer program further causes the processor to generate a plurality of additional binding motifs for a plurality of respective additional MHCs, wherein screening includes screening in accordance with the plurality of additional binding motifs.

13. The system of claim 11, wherein the computer program further causes the processor to train the peptide mutation policy neural network by maximizing an objective function as:
$${max}_{θ} {L}^{CLIP} ( θ ) = {t}_{} [ min ( {r}_{t} ( θ ) {\hat{A}}_{t} , clip ( {r}_{t} ( θ ) , 1 - ϵ , 1 + ϵ ) {\hat{A}}_{t} ) ]$$
where θ represents parameters of the peptide mutation policy neural network, [symbol] ₜ is an expectation with respect to a time step t, rₜ(θ) is a probability ratio between an action under a current policy and an action under a previous policy, Âₜ is an average at time step t, clip(·) is a clipping function, and E is a size of a clipping interval.

14. The system of claim 11, wherein the computer program further causes the processor to pre-train the peptide mutation policy neural network using an expert policy.

15. The system of claim 11, wherein the computer program further causes the processor to determine pairwise Euclidean distances between block substitution matrix (BLOSUM) representations of the plurality of library peptides and a BLOSUM representation of the binding motif.

16. The system of claim 11, wherein the computer program further causes the processor to determine log-likelihood of the plurality of library peptides under a weighted position of the binding motif.

17. The system of claim 11, wherein the computer program further causes the processor to sample a random starting peptide and applying a change to the random starting peptide according to the peptide mutation policy.

18. The system of claim 11, wherein the computer program further causes the processor to change an input peptide sequence as an action and to determine a reward for the action based on the peptide presentation score of the changed input peptide sequence.

19. The system of claim 11, wherein the computer program further causes the processor to compare the screened plurality of library peptides to a candidate vaccine peptide to determine how the candidate vaccine peptide binds to the MHC.

20. The system of claim 19, wherein the computer program further causes the processor to create a vaccine based on the candidate vaccine peptide.
